(19) United States
(12) Patent Application Publication
(10) Pub. No.: US 2006/0094816 A1
(43) Pub. Date: May 4, 2006

(54) Processes for preparing energy-curable emulsions and processes for coating substrates therewith

(76) Inventors: Shailesh Shah, Ambler, PA (US); Anbu Natesh, North Wales, PA (US); Ronald LaFreeda, Harleysville, PA (US); Ramesh Narayan, Harleysville, PA (US); Issac Fleming, Charlotte, NC (US)

Correspondence Address: COGNIS CORPORATION;PATENT DEPARTMENT, 300 BROOKSIDE AVENUE, AMBLER, PA, 19002, US

(21) Appl. No.: 11/121,393

(22) Filed: May 4, 2005

Related U.S. Application Data
(60) Provisional application No. 60/568,337, filed on May 5, 2004.

(51) Int. Cl. A61K 9/16

(52) U.S. Cl. 524/543

(57) ABSTRACT

A process for forming an emulsion of an energy-curable component by passing the energy-curable component, water and optionally a surfactant through an in-line mixing zone. The in-line mixing zone can be a static mixing zone or a high shear in-line mixing zone having a rotor-stator assembly. The emulsion can be prepared contemporaneously (just-in-time) with its use.

### RELATED APPLICATIONS

This application claims priority from provisional application Ser. No. 60/568,337 filed May 5, 2004, the entire contents of which are incorporated herein by reference.

### BACKGROUND OF THE INVENTION

Many energy-curable coating formulations are applied in the form of aqueous emulsions to substrates which are to be coated with the formulations. The solids content of the emulsions vary greatly but are generally within a range of 10% to 80%. Ordinarily, an end user of an energy-curable coating formulation will receive the emulsion from a formulator who passes on the cost of shipping a great deal of water. Additionally, many energy-curable coating emulsions are unstable over time and undergo phase separation and/or precipitation upon storage.

Thus, a need exists in the art for improved processes for the preparation of emulsions of energy-curable coating formulations.

### BRIEF SUMMARY OF THE INVENTION

The present invention is directed to processes for preparing aqueous emulsions of energy-curable coating materials. More specifically, the present invention is directed to highly efficient, in-line processes for preparing emulsions of energy-curable coating materials, which processes allow for the just-in-time preparation of such emulsions and the contemporaneous, or nearly contemporaneous, application of such coating emulsions onto a substrate. The processes for preparing emulsions according to the present invention provide numerous advantages over prior art methods of preparing emulsions of energy curable coating formulations. For example, the in-line, just-in-time preparation of an emulsion in accordance with the present invention allows for the on-site provision of water thus decreasing the shipping costs associated with transporting an energy-curable component and/or a mixture of an energy-curable component and a surfactant. In contrast to prior art emulsions, where the coatings formulator or the end user could only reduce the solids from the level supplied by the emulsion manufacturer, the current invention, allows the formulator and/or the end user to dial-in the formulation to a much broader range of solids content. In addition, the processes according to the present invention provide stable emulsions only in quantities as needed thereby minimizing the contact of organic and aqueous phase and reducing the aging of emulsified but unused coating formulations.

One embodiment of the present invention includes a process for preparing an emulsion, said process comprising: (a) providing an in-line mixing apparatus; and (b) mixing an energy-curable component, an optional surfactant and water with the in-line mixing apparatus to form an emulsion. In certain preferred embodiments of the present invention, the energy-curable component comprises an ethylenically unsaturated compound. In certain preferred embodiments of the present invention, the emulsion is substantially free of components having a high skin irritancy potential.

A preferred embodiment of the present invention includes a process for preparing a just-in-time emulsion, said process comprising: (a) providing a static in-line mixing zone; and (b) mixing (i) a (meth)acrylate oligomer selected from the group consisting of polyether (meth)acrylates, polyester (meth)acrylates, urethane (meth)acrylates, epoxy (meth)acrylates, poly(meth)acrylates, and novalac epoxy resin (meth)acrylates, (ii) a surfactant and (iii) water in the static in-line mixing zone having a high pressure differential across the static in-line mixing zone to form an emulsion. Preferably the emulsion is substantially free of components having a high skin irritancy potential.

Yet another embodiment of the present invention includes a process for coating a substrate, said process comprising: (a) providing an in-line mixing apparatus; (b) mixing an energy-curable component, a surfactant and water with the in-line mixing apparatus to form an emulsion; (c) applying the emulsion to at least a portion of the substrate; and (d) subjecting the emulsion to a source of energy sufficient to form a cured coating.

Yet another embodiment of the present invention includes a process for coating a substrate, said process comprising: (a) providing an in-line mixing apparatus; (b) mixing an energy-curable component, i.e. a reactive surfactant made by grafting a non-ionic surfactant on to a multifunctional epoxide, the said non-ionic surfactant grafted epoxide is then reacted with (meth)acrylic acid, with water from a separate stream in preferably an in-line mixing apparatus to form an emulsion; (c) applying the emulsion to at least a portion of the substrate; and (d) subjecting the emulsion to a source of energy sufficient to form a cured coating.

### DETAILED DESCRIPTION OF THE INVENTION

Other than in the operating examples, or where otherwise indicated, all numbers expressing quantities of ingredients or reaction conditions used herein are to be understood as modified in all instances by the term “about”.

As used herein, the term “(meth)acrylate” shall mean acrylate and/or methacrylate. Similarly, as used herein, the term “(meth)acrylic” shall mean acrylic and/or methacrylic.

The present invention includes processes for preparing emulsions, wherein an in-line mixing apparatus is employed. Numerous different in-line mixing apparatus can be used in accordance with the processes of the present invention. Suitable in-line mixing apparatus for use in accordance with the processes of the present invention include, but are not limited to, static in-line mixers and high shear mixers having a rotor-stator assembly. In certain preferred embodiments of the present invention, the in-line mixing apparatus includes a static in-line mixer. In certain more preferred embodiments of the present invention the in-line mixing apparatus includes a static in-line mixer, and the mixing of the energy-curable component, the surfactant and water is carried out with a high pressure differential across the static mixing zone. As used herein, high pressure differentials refers to pressure differentials greater than 200 psi, more preferably greater than 1000 psi, and most preferably greater than 2500 psi. The high pressure differential increases mixing in the static mixing zone. Preferably the mixing is turbulent mixing.

Static mixers are available from a number of manufacturers. In general, static mixers or motionless mixers comprise fins, obstructions, and/or channels mounted in pipes or other conduits for fluid travel, designed to promote mixing as fluid flows through the mixer. Most static mixers use some method of first dividing the flow, then rotating, channeling, or diverting the flow, before re-combining. Other static mixers create additional turbulence to enhance mixing.

A number of various different static in-line mixers, also known as interfacial surface generators, which may be used in accordance with the processes of the present invention are described in U.S. Pat. Nos. 3,358,749; 3,404,869; 3,652,061; and 3,583,678; the entire contents of each of which is hereby incorporated by reference.

High shear electrically powered in-line mixers having rotor-stator assemblies are also available from a variety of manufacturers.

The process of the invention can provide emulsions of resins having relatively high viscosities which, at times, are difficult to prepare using conventional tank mixing methods.

In certain embodiments of the invention, the energy-curable component, the surfactant and the water can be conveyed via pipes or other suitable conduits from holding tanks or the like via a system of pumps and valves, which may optionally be controlled by a central processing unit. The components should be adequately controlled to provide the required emulsion composition. Suitable schematic designs for such a system are described in U.S. Pat. Nos. 6,251,958 and 6,491,839; the entire contents of each of which is hereby incorporated by reference. In certain preferred embodiments of the processes according to the present invention, an energy-curable component and a surfactant are premixed before introduction into the in-line mixing apparatus with the water.

Various different energy-curable components can be mixed in accordance with the processes of the present invention to form emulsions for the coating of substrates. In various preferred embodiments of the processes according to the present invention, the energy-curable component comprises an ethylenically-unsaturated compound. Ethylenically-unsaturated compounds suitable for use in accordance with the processes of the present invention include (meth)acrylates, (meth)acrylics, vinyl compounds and allylic compounds. Additional ethylenically-unsaturated compounds suitable for use in accordance with the processes of the present invention include (meth)acrylate oligomers, such as for example, polyether (meth)acrylates, polyester (meth)acrylates, urethane (meth)acrylates, epoxy (meth)acrylates, poly (meth)acrylates, and novalac epoxy resin (meth)acrylates.

In certain preferred embodiments of the processes according to the present invention the ethylenically-unsaturated compound comprises a (meth)acrylate functional compound, and more preferably a polyfunctional (meth)acrylate compound.

Suitable polyether (meth)acrylates for use as the energy-curable component in the processes according to the present invention include reaction products of an alcohol and an alkylene oxide, further reacted with an α-β-unsaturated acid. Suitable polyester (meth)acrylates for use as the energy-curable component in the processes according to present invention include reaction products of a polyol, a diacid or anhydride, and (meth)acrylic acid. Suitable epoxy acrylates include reaction products of mono- or poly-epoxides and (meth)acrylic acid. Suitable urethane acrylates include the reaction products of a polyol, a polyisocyanate and a hydroxyalkyl (meth)acrylate. Suitable urethane acrylates also include reaction products of a polyisocyanate, a polyol, and adducts of hydroxyalkyl (meth)acrylates with lactones, as well as reaction products of polyisocyantes, polyols and melamine (meth)acrylates. Other energy-curable components that may be used include amine-modified polyether (meth)acrylates, which are reaction products of a polyether (meth)acrylate as defined above and either less than a stoichiometric amount of a primary amine or up to a stoichiometric amount of a secondary amine.

Various mixtures of two or more energy-curable components can be used as the energy curable component in accordance with the processes of the present invention. Moreover, functional monomers which serve dual purposes can also be included in the energy-curable component to be mixed according to the present invention. For example, the energy-curable component used in the processes according to the present invention may comprise a polyfunctional (meth)acrylate such as a bisphenol A (4) ethoxylate di(meth)acrylate, a urethane (meth)acrylate such as an aliphatic urethane di(meth)acrylate and a monofunctional monomer such as a nonylphenol (4) ethoxylate (meth)acrylate. Certain monofunctional monomers can act as surface-active agents, wetting agents, flow and level modifiers, adhesion promoters, resin compatabilizers, hardness agents, and/or viscosity reducers as well as providing additional energy-curable components.

Suitable surfactants for use in accordance with the present invention include any and all surface active agents capable of emulsifying the energy-curable component and water, and mixtures thereof. Such surfactants include, but are not limited to, nonionic surfactants, anionic surfactants, amphoteric surfactants, zwitterionic surfactants, and cationic surfactants. In addition, certain monofunctional monomers which are capable of providing sufficient surface activity to emulsify an energy-curable component and water may be used as a surfactant, or in addition to another surfactant, in accordance with the processes of the present invention. In certain preferred embodiments of the present invention, the surfactant comprises a nonionic or anionic surfactant. More preferably, a surfactant for use in accordance with the present invention will comprise a nonionic surfactant.

The water used in the processes according to the present invention may be from any water source. In certain preferred embodiments of the present invention the water is distilled. Even more preferably the water used is distilled and deionized water.

The coating emulsions according to the present invention may further contain reactive diluents, photoinitiators, pigments, additional adhesion promoters, flatting agents, stabilizers and other additives and auxiliaries generally incorporated into coating formulations. Preferably the additives and auxiliaries are mixed with the energy curable component before passing through the in-line mixing zone for emulsification with water.

In certain preferred embodiments of the present invention, the emulsions prepared will be substantially free of components which have a high skin irritancy potential. Such components include certain functional monomers that are often included in energy-curable coating emulsions to reduce viscosity. As used herein, a high skin irritancy potential refers to a composition or compound having a score of 3 or higher on the primary irritation index scale of 1 to 7. Moreover, as used herein, “substantially free”shall mean that less than about 10% by weight of the emulsion is comprised of such components. More preferably, less than about 5% by weight of the emulsion is comprised of such components. Most preferably, less than about 1% by weight of the emulsion is comprised of such components.

The emulsions prepared by the processes according to the present invention can have a solids content ranging from about 1% to 99%, but will preferably have a solids content of from 10 to 80%, more preferably 20 to 70% and most preferably 25 to 55%. The amounts of energy-curable component and surfactant may vary widely depending upon the specific end use of the emulsion. In general, the emulsion can contain from about 1 to about 20% of a surfactant. The remainder of the non-aqueous portion can be entirely comprised of the energy-curable component, or may comprise the energy-curable component and other suitable additives or auxiliaries.

The present invention also relates to processes for coating substrates. Virtually any substrate can be coated via the process according to the present invention, based upon the selection of proper coating materials and additives, as known by those of ordinary skill in the art. Examples of suitable substrates include woods, metals, glass, plastics, and textiles. The emulsions prepared by the processes according to the present invention can be applied in the coating processes according to the present invention in any number of ways. The particular method of application is not critical. The emulsions may be applied via spraying, dipping, rolling, or any other suitable method of contacting the substrate with the emulsion.

The source of energy to which the substrate-applied emulsion is subjected may be any energy source capable of curing the energy-curable component, and can include radiant heat, UV radiation and/or electron beam bombardment. In those preferred embodiments of the present invention wherein the energy-curable component comprises an ethylenically unsaturated component, the preferred source of energy to which the substrate-applied emulsion is subjected comprises UV radiation and/or electron beam bombardment. Generally the coating is dried before the energy is applied to cure the coating.

In certain preferred embodiments of the processes for coating a substrate in accordance with the present invention, the application of the emulsion to at least a portion of the substrate occurs within a period of time after the emulsion is formed such that no phase separation or settling occurs within the emulsion. More preferably, the application will occur within several days of the emulsion being formed. Even more preferably, the application will occur within forty-eight hours of the emulsion being formed. Even more preferably, the application will occur within several hours of the emulsion being formed. Most preferably, there will be virtually no delay between formation of the emulsion and application thereof to the substrate. In various preferred embodiments, application and curing both occur within the time periods set forth above.

The present invention will now be illustrated in more detail by reference to the following specific, non-limiting example.

### EXAMPLE 1

A formulation was prepared containing: Photomer® 6210, an acrylated aliphatic urethane available from Cognis Corp.; Photomer® 4028, a bisphenol-A ethoxylated diacrylate; and a surfactant; wherein the Photomer® 6210 and 4028 are blended in a ratio by weight of 2:3. The surfactant was added in an amount of about 10% by weight of the oligomer. This formulation was pumped into a static in-line mixer at the same time water was pumped into the mixer. The formulation and the water were passed through the in-line mixer under high pressure. The resulting emulsion was ready-to-use.

The formulation was prepared by mixing 40 grams of PHOTOMER® 4020 in a jar. The jar was heated in an oven at 60° C. for one hour. The mixture was stirred with a spatula until the mixture was clear and uniform. There was some loss of material. Ten grams of HYDROPLAT® 65 were added to the mixture and stirred slowly to form a uniform opaque milky liquid. The jar was placed in an oven at 60° C. for one hour. The mixture became clear. The sample was cooled to room temperature and the sample became a milky liquid.

Three emulsions containing 50%, 60% and 70% solids by weight based on the weight of the emulsion were prepared using a mixture as prepared above. The properties of the emulsion are set out below.

| | Percent | Type | | | Viscosity |
|---|---|---|---|---|---|
| Lot # | Solids | Viscosimeter | Spindle | RPM | 25° C. CP |
| A | 50 | LVT | 2 | 60 | 245 |
| B | 60 | RVT | 2 | 10 | 3280 |
| C | 70 | RVT | 4 | 10 | 38000 |

Lot A was drawn down to a 10 micron coating on a phosphate coated steel panel. The coated panel was heated at 60° C. for four minutes to dry the coating. The coating was cured using an electron beam which applied 30 K Grays of radiation. The coating had a 5 B crosshatch adhesion.

What is claimed is:

1. A process for preparing an emulsion, said process comprising:
(a) providing an in-line mixing zone; and
(b) mixing an energy-curable component, optionally a surfactant and water in the in-line mixing zone to form an emulsion.

2. The process according to claim 1, wherein the energy-curable component and the surfactant are premixed.

3. The process according to claim 1, wherein the energy-curable component comprises an ethylenically unsaturated compound.

4. The process according to claim 1, wherein the energy-curable component comprises a compound selected from the group consisting of (meth)acrylics, (meth)acrylates, vinyl compounds and allyl compounds.

5. The process according to claim 1, wherein the energy-curable component comprises a (meth)acrylate-functional compound.

6. The process according to claim 1, wherein the energy-curable component comprises a polyfunctional (meth)acrylate.

7. The process according to claim 1, wherein the energy-curable component comprises a (meth)acrylate oligomer selected from the group consisting of polyether (meth)acrylates, polyester (meth)acrylates, urethane (meth)acrylates, epoxy (meth)acrylates, poly(meth)acrylates, novalac epoxy resin (meth)acrylates, melamine (meth)acrylates and (meth)acrylate functional (meth)acrylic resins.

8. The process according to claim 1, wherein the energy-curable component comprises an epoxy (meth)acrylate oligomer.

9. The process according to claim 1, wherein the surfactant comprises a nonionic surfactant.

10. The process according to claim 1, wherein the in-line mixing zone comprises a static in-line mixer.

11. The process according to claim 1, wherein the in-line mixing zone comprises a high shear in-line mixer having a rotor-stator assembly.

12. The process according to claim 1, wherein the energy-curable component, the surfactant, and water are mixed at a high pressure differential of at least 200 psi across a static mixing zone.

13. The process according to claim 12, wherein the energy-curable component, the surfactant, and water are mixed at a pressure differential of at least 1000 psi across the static mixing zone.

14. The process according to claim 1, wherein the emulsion is substantially free of components having a high skin irritancy potential.

15. The process according to claim 14, wherein the emulsion comprises an amount of components having a high skin irritancy potential of less then 10% by weight.

16. A process for preparing a just-in-time emulsion, said process comprising:
(a) providing a static in-line mixing zone; and
(b) mixing (i) a (meth)acrylate oligomer selected from the group consisting of polyether (meth)acrylates, polyester (meth)acrylates, urethane (meth)acrylates, epoxy (meth)acrylates, poly(meth)acrylates, and novalac epoxy resin (meth)acrylates, (ii) a surfactant and (iii) water in the static in-line mixing zone at a pressure differential across the mixing zone of at least 200 psi to form an emulsion.

17. A process for coating a substrate, said process comprising:
(a) providing an in-line mixing zone;
(b) mixing an energy-curable component, a surfactant and water in the in-line mixing zone to form an emulsion;
(c) applying the emulsion to at least a portion of the substrate; and
(d) subjecting the emulsion to a source of energy sufficient to form a cured coating.

18. The process according to claim 17, wherein the emulsion is applied to at least the portion of the substrate within a period of time after the emulsion is formed such that no phase separation or settling occurs within the emulsion.

19. The process according to claim 17, wherein the energy-curable component comprises an ethylenically unsaturated compound.

20. The process according to claim 17, wherein the in-line mixing zone comprises a static in-line mixer.

21. The process of claim 1 wherein the surfactant is present in a range of 1% up to 10% by weight of the energy-curable component.
